(19) United States
(12) Patent Application Publication
(10) Pub. No.: US 2021/0228746 A1
(43) Pub. Date: Jul. 29, 2021

(54) Affinity Magnetic Particles For Imaging System

(71) Applicant: Board of Trustees of Michigan State University, East Lansing, MI (US)

(72) Inventors: Erik M. SHAPIRO, Okemos, MI (US); Shatadru CHAKRAVARTY, Okemos, MI (US)

(73) Assignee: Board of Trustees of Michigan State University, East Lansing, MI (US)

(21) Appl. No.: 17/158,332

(22) Filed: Jan. 26, 2021

Related U.S. Application Data
(60) Provisional application No. 62/966,414, filed on Jan. 27, 2020.

(51) Int. Cl. A61K 49/18

(52) U.S. Cl. CPC A61K 49/1896; A61K 49/1857; A61K 49/1875; A61K 49/1833

(57) ABSTRACT

A nanoparticle construct is provided. The nanoparticle construct includes a nanoparticle defining an outer surface, a magnetic nanocrystal carried by the nanoparticle, and a coupling agent extending from the outer surface of the nanoparticle. The coupling agent is configured to couple the nanoparticle construct to a cell.

### CROSS-REFERENCE TO RELATED APPLICATIONS

This application claims the benefit of U.S. Provisional Application No. 62/966,414, filed on Jan. 27, 2020. The entire disclosure of the above application is incorporated herein by reference.

### FIELD

The present disclosure relates to coupling magnetic nanoparticles to cells for magnetic resonance imaging and magnetic particle imaging.

### BACKGROUND

This section provides background information related to the present disclosure which is not necessarily prior art.

Immunotherapy is a medical treatment where a patient's immune system is activated to treat diseases such as cancer. Chimeric antigen receptor T cell therapy, or “CAR-T cell therapy,” represents a promising therapy within this category. In CAR-T cell therapy, a patient's own isolated T cells are genetically modified to produce chimeric antigen receptors (CARs). The CARs are both receptors that bind to a specific cancer cell antigen and activators that activate T cells. After they are generated, the CAR-T cells are administered back into the patient. Accordingly, the CAR-T cells are capable of both targeting only cancer cells that express the antigen by way of the CARs' receptor functionality and destroying the cancer cells by way of the CARs' T cell-activating functionality.

Unfortunately, the efficacy of CAR-T cell therapy can only be assessed three months after treatment, as physicians must wait a sufficient time for the treatment to significantly affect the disease. Additionally, CAR-T cells have been linked to severe side effects, such as neurotoxicity and cytokine release syndrome, which must be continually monitored for the first two months. Multiple deaths in late-stage clinical trials with CAR-T therapy have been documented. Therefore, the use of CAR-T cells for cancer therapy requires the means to track the delivery and migration of the CAR-T cells to both the desired tumor location and to potentially off-target sites.

### SUMMARY

This section provides a general summary of the disclosure, and is not a comprehensive disclosure of its full scope or all of its features.

The current technology provides affinity magnetic particles for imaging systems.

In various aspects, the current technology provides a nanoparticle construct including a nanoparticle defining an outer surface, a magnetic nanocrystal carried by the nanoparticle, and a coupling agent extending from the outer surface of the nanoparticle, wherein the coupling agent is configured to couple the nanoparticle construct to a cell.

In one aspect, the magnetic nanocrystal includes a metal, a metal oxide, a metal alloy, or combinations thereof.

In one aspect, the nanoparticle includes a polymer matrix and the magnetic nanocrystal is embedded within the polymer matrix.

In one aspect, the polymer matrix includes poly(lactic-co-glycolic acid) (PLGA) and optionally polylactic acid (PLA), poly(caprolactone), or combinations thereof.

In one aspect, the magnetic nanocrystal is embedded within the polymer matrix.

In one aspect, the coupling agent is a maleimide functionality, an antibody, an antibody fragment, or combinations thereof.

In one aspect, the polymer matrix includes PLGA and the coupling agent includes the maleimide functionality, wherein the maleimide functionality is coupled to the PLGA by way of a linker.

In one aspect, the linker is polyethylene glycol (PEG).

In one aspect, the nanoparticle construct is coupled to a T cell by way of a bond between the maleimide functionality and a thiol group on the T cell's cell membrane.

In one aspect, the nanoparticle construct is synthesized by a method including dissolving a polymer and the coupling agent in an organic solvent to form a polymer solution, adding a dispersion including the magnetic nanocrystal to the polymer solution to form a precursor solution, transferring the precursor solution to a solution including poly-vinyl alcohol (PVA) and water to form a nanoparticle precursor solution, sonicating or homogenizing the nanoparticle precursor solution, and removing the organic solvent to form the nanoparticle construct.

In one aspect, the nanoparticle includes a substantially spherical lipid membrane including a plurality of lipids that define an interior compartment, the magnetic nanocrystal is disposed within the interior compartment, and the coupling agent is bonded to at least one lipid of the plurality.

In one aspect, the coupling agent is a maleimide functionality, an antibody, an antibody fragment, or combinations thereof.

In one aspect, the nanoparticle construct is coupled to a T cell by way of the coupling agent.

In one aspect, the coupling agent includes the maleimide functionality and the maleimide functionality is bonded to a thiol group on the T cell's cell membrane.

In one aspect, the nanoparticle construct is synthesized by a method including adding water to a dispersion including chloroform, lipids, at least one coupling agent-functionalized lipid, and the magnetic nanocrystal to form an emulsion, sonicating the emulsion, adding additional water to the emulsion and sonicating to form a water-in-oil-in-water double emulsion, and removing the chloroform from the water-in-oil-in-water double emulsion to form the nanoparticle construct.

In various aspects, the current technology also provides a method of detecting a cell in a subject, the method including subjecting the subject to magnetic resonance imaging (MRI) or magnetic particle imaging (MPI), visualizing the cell in a resulting MRI scan or MPI scan, and determining a location of the cell in the subject, wherein the cell was previously isolated from the subject, coupled to the nanoparticle construct, and administered back to the subject.

In one aspect, the cell is a T cell.

In one aspect, the subject has cancer and the T cell is a T cell including a chimeric antigen receptor (CAR-T cell).

In various aspects, the current technology also provides a method of treating a subject in need thereof, the method including subjecting the subject, or having the subject subjected to, a treatment including isolating a cell from the subject; modifying the cell to generate a therapeutic cell; coupling the therapeutic cell to a nanoparticle construct to form a magnetic therapeutic cell, the nanoparticle construct including a nanoparticle defining an outer surface, a magnetic nanocrystal carried by the nanoparticle, and a coupling agent extending from the outer surface of the nanoparticle, wherein the coupling agent is configured to couple the nanoparticle construct to the therapeutic cell; and administering the magnetic therapeutic cell to the subject, performing magnetic resonance imaging (MRI) or magnetic particle imaging (MPI) on the subject, determining the location of the magnetic therapeutic cell based on a MRI image or a MPI image, and when the location of the magnetic therapeutic cell is determined to be at a location needing the therapeutic cell, continuing the treatment, or when the location of the magnetic therapeutic cell is determined to be at a location other than a location needing the therapeutic cell, discontinuing the treatment.

In one aspect, the nanoparticle includes either a polymer matrix or a substantially spherical lipid membrane defining an interior compartment carrying the magnetic nanocrystal.

In one aspect, the coupling agent is a maleimide functionality, an antibody, an antibody fragment, or combinations thereof.

In various aspects, the current technology also provides a method of treating a subject in need thereof, the method including performing magnetic resonance imaging (MRI) or magnetic particle imaging (MPI) on the subject, wherein the subject is undergoing a treatment in which a cell was isolated from the subject; the cell was modified to generate a therapeutic cell; the therapeutic cell was coupled to a nanoparticle construct a magnetic therapeutic cell, the nanoparticle construct including a nanoparticle defining an outer surface, a magnetic nanocrystal carried by the nanoparticle, and a coupling agent extending from the outer surface of the nanoparticle and bonded to the therapeutic cell; and the magnetic therapeutic cell was administered to the subject, determining the location of the magnetic therapeutic cell based on a MRI image or a MPI image, and when the location of the magnetic therapeutic cell is determined to be at a location needing the therapeutic cell, continuing the treatment, or when the location of the magnetic therapeutic cell is determined to beat a location other than a location needing the therapeutic cell, discontinuing the treatment.

In one aspect, the nanoparticle includes either a polymer matrix or a substantially spherical lipid membrane defining an interior compartment carrying the magnetic nanocrystal and the coupling agent is a maleimide functionality, an antibody, an antibody fragment, or combinations thereof.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a graphic illustration of a nanoparticle construct in accordance with various aspects of the current technology.

FIG. 2 is a graphic illustration of a nanoparticle construct including a PLGA nanoparticle having magnetic nanocrystals embedded therein and coupling agents extending from the PLGA nanoparticle in accordance with various aspects of the current technology.

FIG. 3A is a graphic illustration of the nanoparticle construct of FIG. 2 in which the coupling agents are antibodies in accordance with various aspects of the current technology.

FIG. 3B is a graphic illustration of the nanoparticle construct of FIG. 3A bound to a cell in accordance with various aspects of the current technology.

FIG. 4A is a graphic illustration of the nanoparticle construct of FIG. 2 in which the coupling agents are maleimide functionalities in accordance with various aspects of the current technology.

FIG. 4B is a graphic illustration of the nanoparticle construct of FIG. 4A bound to a cell in accordance with various aspects of the current technology.

FIG. 5 is a graphic illustration of a nanoparticle construct including a spherical lipid membrane nanoparticle having magnetic nanocrystals disposed therein and coupling agents extending from the spherical lipid membrane nanoparticle in accordance with various aspects of the current technology.

FIG. 6A is a graphic illustration of the nanoparticle construct of FIG. 5 in which the coupling agents are antibodies in accordance with various aspects of the current technology.

FIG. 6B is a graphic illustration of the nanoparticle construct of FIG. 6A bound to a cell in accordance with various aspects of the current technology.

FIG. 7A is a graphic illustration of the nanoparticle construct of FIG. 5 in which the coupling agents are maleimide functionalities in accordance with various aspects of the current technology.

FIG. 7B is a graphic illustration of the nanoparticle construct of FIG. 7A bound to a cell in accordance with various aspects of the current technology.

FIG. 8 is an illustration showing an exemplary method of making an exemplary nanoparticle construct in accordance with various aspects of the current technology.

FIG. 9A is a fluorescence micrograph showing exemplary nanoparticle constructs bound to a fluorescent thiol molecule, wherein the nanoparticle constructs comprise 10% iron oxide and 10% maleimide in accordance with various aspects of the current technology. The scale bar is 500 μm.

FIG. 9B is a fluorescence micrograph showing exemplary nanoparticle constructs bound to a fluorescent thiol molecule, wherein the nanoparticle constructs comprise 10% iron oxide and 50% maleimide in accordance with various aspects of the current technology. The scale bar is 500 μm.

FIG. 9C is a fluorescence micrograph showing exemplary nanoparticle constructs bound to a fluorescent thiol molecule, wherein the nanoparticle constructs comprise 10% iron oxide and 100% maleimide in accordance with various aspects of the current technology. The scale bar is 500 μm.

FIG. 9D is a fluorescence micrograph showing exemplary nanoparticle constructs bound to a fluorescent thiol molecule, wherein the nanoparticle constructs comprise 25% iron oxide and 10% maleimide in accordance with various aspects of the current technology. The scale bar is 500 μm.

FIG. 9E is a fluorescence micrograph showing exemplary nanoparticle constructs bound to a fluorescent thiol molecule, wherein the nanoparticle constructs comprise 25% iron oxide and 50% maleimide in accordance with various aspects of the current technology. The scale bar is 500 μm.

FIG. 9F is a fluorescence micrograph showing exemplary nanoparticle constructs bound to a fluorescent thiol molecule, wherein the nanoparticle constructs comprise 25% iron oxide and 100% maleimide in accordance with various aspects of the current technology. The scale bar is 500 μm.

FIG. 10A is a transmission electron microscopy (TEM) micrograph of a nanoparticle construct comprising 50% iron oxide and 10% maleimide in accordance with various aspects of the current technology. The scale bar is 50 nm.

FIG. 10B a scanning electron microscopy (SEM) micrograph of nanoparticle constructs comprising 50% iron oxide, 10% maleimide, and 20% FITC in accordance with various aspects of the current technology. The scale bar is 500 nm.

FIG. 11A is a bright field micrograph showing nanoparticle constructs bound to Jurkat cells at a first magnification in accordance with various aspects of the current technology.

FIG. 11B is a bright field micrograph showing nanoparticle constructs bound to Jurkat cells at a second higher magnification in accordance with various aspects of the current technology.

FIG. 12A shows fluorescence and bright field micrographs of a Jurkat cell conjugated with nanoparticle constructs prepared in accordance with various aspects of the current technology.

FIG. 12B shows fluorescence and bright field micrographs of Jurkat cells conjugated with control nanoparticle constructs that do not include maleimide.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

The current technology provides nanoparticle constructs that carry magnetic nanocrystals. The nanoparticle constructs can be coupled to a specific and desired cell type to form a magnetically labeled cell. After the magnetically labeled cell is introduced to a subject, its location within the subject can be determined by magnetic resonance imaging (MRI) or magnetic particle imaging (MPI). This imaging enables physicians to rapidly determine the exact location of injected cells for accurate targeting of, for example, solid tumors. Thus, treatment efficacy can be assessed on the time scale of days, rather than months. If cells have not arrived to the desired tumor, the subject can rapidly be switched to another therapy. Further, a smaller number of cells can be injected to see if they home to the sites of tumors. If the cells do not localize to tumors, then a larger injection of cells may not be warranted, cutting down on the cost of potential therapy. With the current cost of CAR-T therapy being between $375,000 and $475,000 per patient, this technology has enormous impact on cost of care. The subject or patient described herein can be any human or non-human mammal.

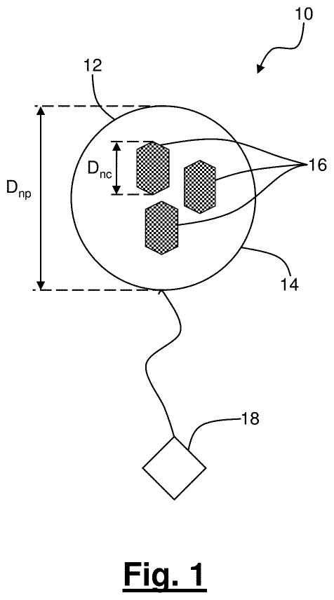

With reference to FIG. 1, the current technology provides a nanoparticle construct 10. The nanoparticle construct 10 comprises a nanoparticle 12 having an outer surface 14 and a magnetic nanocrystal 16 (or at least one magnetic nanocrystal 16, i.e., a plurality of magnetic nanocrystals 16) carried by the nanoparticle 12. By “carried by the nanoparticle,” it is meant that the magnetic nanocrystal 16 is physically associated with the nanoparticle 12 such that the nanoparticle 12 and the magnetic nanocrystal 16 are substantially inseparable under normal conditions. The nanoparticle 12 has a nanoparticle diameter Dₙₚ of greater than or equal to about 100 nm to less than or equal to about 2000 nm or greater than or equal to about 200 nm to less than or equal to about 1000 nm. The diameter Dₙₚ can be, for example, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, about 1250 nm, about 1500 nm, about 1750 nm, or about 2000 nm.

The magnetic nanocrystal 16 comprises a magnetic metal, a magnetic metal oxide, a magnetic metal alloy, or combinations thereof. Non-limiting examples of magnetic metals include gold (Au), silver (Ag), iron (Fe), cobalt (Co), nickel (Ni), and combinations thereof. Non-limiting examples of magnetic metal oxides include oxides of iron (e.g., Fe₃O₄ (magnetite) and Fe₂O₃ (maghemite)), aluminum (Al), titanium (Ti), zinc (Zn), nickel (Ni), copper (Cu), tin (Sn), cobalt (Co), magnesium (Mg), cerium (Ce), bismuth (Bi), yttrium (Y), gadolinium (Gd), and combinations thereof. Non-limiting examples of magnetic metal alloys include alloys comprising at least one of iron (Fe), nickel (Ni), cobalt (Co), aluminum (Al), neodymium (Nd), and combinations thereof, e.g., FeCo, FePt, CoPt, CoFeGa, CuNi, and combinations thereof. The magnetic nanocrystal 16 has a nanoparticle diameter D_{nc} of greater than or equal to about 0.5 nm to less than or equal to about 50 nm or greater than or equal to about 1 nm to less than or equal to about 25 nm. The diameter D_{nc} can be, for example, about 0.5 nm, about 0.6 nm, about 0.7 nm, about 0.8 nm, about 0.9 nm, about 1 nm, about 2 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 30 nm, about 35 nm, about 40 nm, about 45 nm, or about 50 nm.

The nanoparticle construct 10 includes the magnetic nanocrystal 16 at a concentration of greater than or equal to about 30 wt. % to less than or equal to about 80 wt. % (based on the total weight of the nanoparticle construct 10), including concentrations of about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, or about 80 wt. %.

The nanoparticle construct 10 yet further comprises a coupling agent 18 extending from the outer surface 14 of the nanoparticle 12. The coupling agent 18 is configured to bind to a cell, thus making the cell imageable by MRI or MPI. For example, the nanoparticle construct 10 can be coupled to a cell isolated from a subject and then administered back into the subject so that the location of the cell within the subject can be determined by analyzing MRI or MPI images. In certain aspects, the nanoparticle construct 10 comprises at least one, i.e., a plurality of, coupling agents 18. For example, the nanoparticle construct 10 comprises greater than or equal to 1 to less than or equal to about 1000 coupling agents 18.

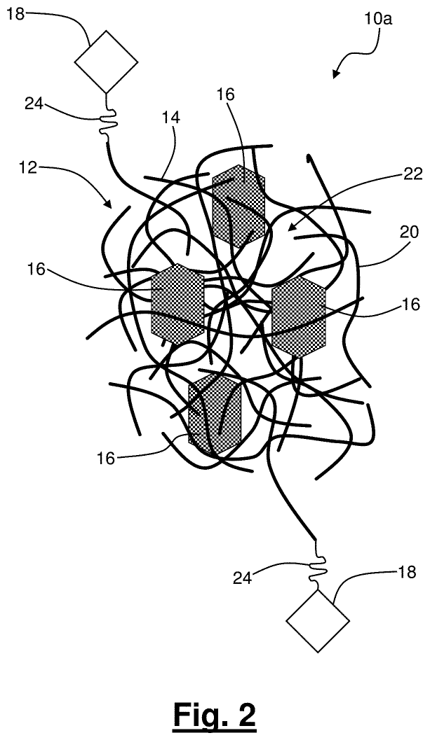

FIG. 2 shows a nanoparticle construct 10a, which is similar to the nanoparticle construct 10 of FIG. 1, but in which the nanoparticle 12 comprises a plurality of polymers 20 arranged in an integrated polymer network defining a polymer matrix 22. The magnetic nanocrystals 16 are embedded within the polymer matrix 22. As such, some of the magnetic nanocrystals 16 may be completed embedded within the polymer matrix 22 and some of the magnetic nanocrystals 16 may be partially embedded within the polymer matrix 22, such that a portion of the magnetic nanocrystals 16 are exposed at the outer surface 14 of the nanoparticle 12.

The plurality of polymers 20 comprise poly(lactic-co-glycolic acid) (PLGA) and optionally polylactic acid (PLA), poly(caprolactone) (polyester), derivatives thereof, or combinations thereof. A portion of the PLGA is modified with a linker 24 that links the PLGA to the coupling agent 18.

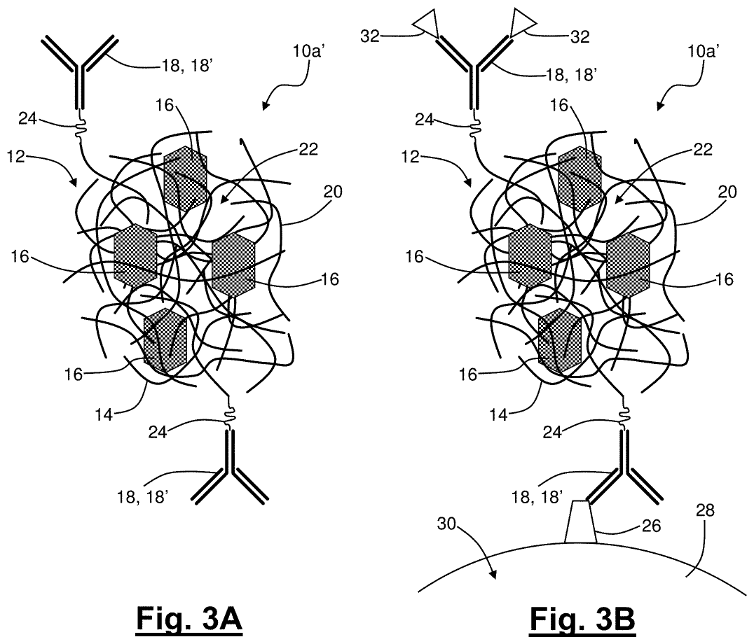

FIG. 3A shows a nanoparticle construct 10a′, which is similar to the nanoparticle construct 10a of FIG. 2, but in which the coupling agent 18 is an antibody or antibody fragment 18′ that binds to a particular cell surface protein or peptide. The antibody or antibody fragment 18′ is linked to the portion of the PLGA by way of the linker 24, which may be a conjugation peptide or PEG (which can have various chain lengths and molecular weights), as non-limiting examples. In some aspects, the antibody or antibody fragment 18′ is a polyclonal or monoclonal antibody that selectively binds to a protein or peptide that is selectively expressed on a cell of interest, such as on a particular cancer cell. In certain other aspects, the antibody or antibody fragment 18′ is an antibody fragment, such as, for example, Fab, Fab′, Fab₂, Fab′₂, Fd, Fd′, scFv, scFv₂, dAb, or combinations thereof, or a chimeric antibody fragment fusion molecule, wherein the antibody fragment or the chimeric antibody fragment fusion molecule selectively binds to a protein that is expressed on a cell of interest, such as on a particular cancer cell. In yet other aspects, the antibody or antibody fragment 18′ selectively binds to a protein or peptide that is not selectively expressed on the cell of interest. Because the cell of interest is isolated from a subject, the nanoparticle construct 10a′ is only coupled to the cell of interest.

FIG. 3B shows the nanoparticle construct 10a′ wherein the antibody or antibody fragment 18′ is bound to a protein or peptide 26 on a surface 28 of an isolated cell 30, which may be a T cell or a CAR-T cell. Here, the isolated cell 30 is magnetically labeled. Optionally, unbound antibodies or antibody fragments 18′ on the nanoparticle construct 10a′ are quenched by a quenching agent 32 to ensure that the unbound antibodies or antibody fragments 18′ do not bind nonspecifically to undesired cells after administering to a subject. Here, the quenching agent 32 can be a peptide that binds to the antibody or antibody fragment 18′ or a second antibody that that specifically recognizes and binds to the antibody or antibody fragment 18′.

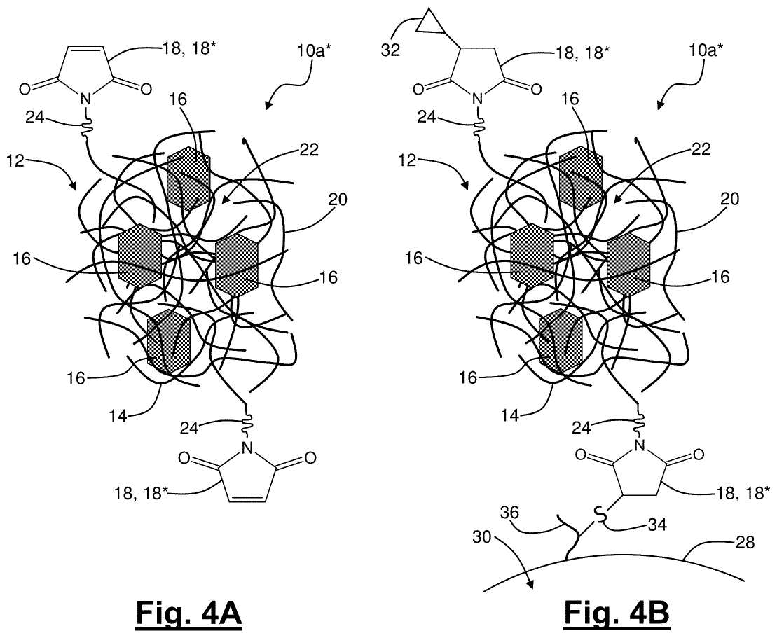

FIG. 4A shows a nanoparticle construct 10a*, which is similar to the nanoparticle construct 10a of FIG. 2, but in which the coupling agent 18 is a maleimide functionality 18* that is linked to a portion of the PLGA by way of the linker 24, which may be PEG (which can have various chain lengths and molecular weights), as a non-limiting example. Accordingly, the nanoparticle construct 10a* can include PLGA-PEG-maleimide constructs. The maleimide functionality 18* binds to thiol groups present on proteins and peptides that are expressed on cell surfaces. Typically, the thiol groups are present in cysteines present within cell surface proteins or peptides.

FIG. 4B shows the nanoparticle construct 10a* wherein the maleimide functionality 18* is bound to a cysteine thiol 34 found in a protein or peptide 36 extending from the surface 28 of the isolated cell 30, which may be a T cell or a CAR-T cell. Here, the isolated cell 30 is magnetically labeled. Optionally, unbound maleimide functionalities 18* on the nanoparticle construct 10a* are quenched by the quenching agent 32 to ensure that the unbound maleimide functionalities 18* do not bind nonspecifically to undesired cells after administering to a subject. Here, the quenching agent 32 can be a thiolated molecule, such as thiolated PEG, a thiolated amino acid, e.g., thiolated glycine, or combinations thereof, as non-limiting examples.

The nanoparticle constructs 10a′, 10a* of FIGS. 3A and 4A, respectively, are made by a method in accordance with the current technology. The method comprises dissolving PLGA, PLGA-Linker-coupling agent (such as PLGA-PEG-maleimide or PLGA-conjugator-antibody), and optionally PLA in an organic solvent to form a polymer solution The organic solvent can be dichloromethane, methoxybenzene (anisole), tetrahydrofuran (THF), ethyl acetate, diethyl ether, methylene chloride, carbon tetrachloride, chloroform, toluene, benzene, cyclohexane, hexane, pentane, and combinations thereof, as non-limiting examples. The PLGA (and PLA when present) and PLGA-Linker-coupling agent are combined in the organic solvent at a PLGA (and optionally PLA) to PLGA-Linker-coupling agent ratio of from about 5:1 to about 20:1, including ratios of about 5:1, about 7.5:1, about 10:1, about 12.5:1, about 15:1, about 17.5:1, and about 20:1. The PLGA, PLGA-Linker-coupling agent, and optional PLA have a concentration of greater than or equal to about 20 mg/mL to less than or equal to about 50 mg/mL in the organic solvent, including concentrations of about 20 mg/mL, about 25 mg/mL, about 30 mg/mL, about 40 mg/mL, about 45 mg/mL, and about 50 mg/m L.

The method further comprises adding a dispersion comprising the magnetic nanocrystal to the polymer solution to form a precursor solution in an oil phase. The dispersion has a magnetic nanocrystal concentration of greater than or equal to about 25 mg/mL to less than or equal to about 40 mg/m L. Greater than or equal to about 0.05 mL to less than or equal to about 0.075 mL of the dispersion is added per mL of the polymer solution to yield a nanocrystal concentration in the precursor solution of greater than or equal to about 0.25 mg/mL to less than or equal to about 5 mg/m L.

The method then comprises transferring the precursor solution to a solution comprising poly-vinyl alcohol (PVA) and water to form a nanoparticle precursor solution. The solution comprising PVA and water comprises greater than or equal to about 1 w/v % to less than or equal to about 5 w/v % PVA in water. The volume of the solution comprising PVA and water is greater than or equal to about 9 mL to less than or equal to about 10 mL per mL of the precursor solution. The method also comprises stirring the nanoparticle precursor solution at a temperature of about 37° C. for a time of from about 1 hour to about 5 hours, including times of about 1 hour, about 2 hours, about 3 hours, about 4 hours, about 5 hours, and times therebetween.

The method then comprises either tip sonicating or homogenizing the nanoparticle precursor solution and removing the organic solvent from the precursor solution to form the nanoparticle construct, which can be collected by centrifugation, washed with deionized water, and optionally frozen and lyophilized.

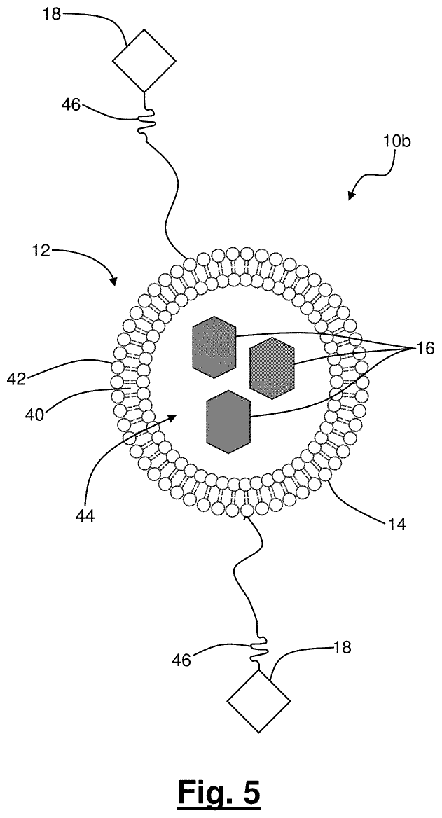

FIG. 5 shows a nanoparticle construct 10b, which is similar to the nanoparticle construct 10 of FIG. 1, but in which the nanoparticle 12 comprises a substantially spherical lipid membrane 40 comprising a plurality of lipids 42. The lipid membrane 40 is a bilayer that defines the outer surface 14 and an interior compartment 44. By “substantially spherical” it is meant that the lipid membrane forms a sphere-like shape, but which may not be a perfect sphere. For example, the substantially spherical lipid membrane 40 may include flat edges or bulges. In various aspects, the lipid membrane 40 is a liposome (also referred to as a “unilamellar vesicle”) or a multi-lamellar liposome (also referred to as a “multi-lamellar vesicle”). The magnetic nanocrystal 16 (or plurality of magnetic nanocrystals 16) are disposed within the interior compartment 44 of the lipid membrane 40. A portion of the plurality of lipids 42 are optionally modified with a linker 46 that links the lipid to the coupling agent 18.

The plurality of lipids can include any amphipathic lipid molecule known in the art capable of forming a liposome. Non-limiting examples of such molecules include phophtaidylcholines, lysophosphatidylcholines, phosphatidic acids, phosphatidylethanolamines, phophatidylglycerols, phosphatidylserines, phosphoinositides, phosphosphigolipids, and combinations thereof.

Non-limiting examples of phosphatidylcholines include 1,2-Didecanoyl-sn-glycero-3-phosphocholine (DDPC), 1,2-Dierucoyl-sn-glycero-3-phosphocholine (DEPC), 1,2-Dilinoleoyl-sn-glycero-3-phosphocholine (DLOPC), 1,2-Dilauroyl-sn-glycero-3-phosphocholine (DLPC), 1,2-Dimyristoyl-sn-glycero-3-phosphocholine (DMPC), 1,2-Dioleoyl-sn-glycero-3-phosphocholine (DOPC), 1,2-Dipalmitoyl-sn-glycero-3-phosphocholine (DPPC), 1,2-Distearoyl-sn-glycero-3-phosphocholine (DSPC), Egg-PC (EPC), Hydrogenated Egg PC (HEPC), High purity Hydrogenated Soy PC (HSPC), Hydrogenated Soy PC (HSPC), 1-Myristoyl-2-palm itoyl-sn-glycero 3-phosphocholine (Milk Sphingomyelin MPPC), 1-Myristoyl-2-stearoyl-sn-glycero-3-phosphocholine (MS PC), 1-Palmitoyl-2-myristoyl-sn-glycero-3-phosphocholine (PMPC), 1-Palm itoyl-2-oleoyl-sn-glycero-3-phosphocholine (POPC), 1-Palm itoyl-2-stearoyl-sn-glycero-3-phosphocholine (PSPC), 1-Stearoyl-2-myristoyl-sn-glycero-3-phosphocholine (SMPC), 1-Stearoyl-2-oleoyl-sn-glycero-3-phosphocholine (SOPC), 1-Stearoyl-2-palmitoyl-sn-glycero-3-phosphocholine (SPPC), and combinations thereof.

Non-limiting examples of lysophosphatidylcholines include 1-Myristoyl-sn-glycero-3-phosphocholine (LYSOPC MYRISTIC), 1-Palm itoyl-sn-glycero-3-phosphocholine (LYSOPC PALMITIC), 1-Stearoyl-sn-glycero-3-phosphocholine (LYLSOPC STEARIC), and combinations thereof.

Non-limiting examples of phosphatidic acids include 1,2-Dierucoyl-sn-glycero-3-phosphate (Sodium Salt) (DEPA-NA), 1,2-Dilauroyl-sn-glycero-3-phosphate (Sodium Salt) (DLPA-NA), 1,2-Dimyristoyl-sn-glycero-3-phosphate (Sodium Salt) (DMPA-NA), 1,2-Dioleoyl-sn-glycero-3-phosphate (Sodium Salt) (DOPA-NA), 1,2-Dipalmitoyl-sn-glycero-3-phosphate (Sodium Salt) (DPPA-NA), 1,2-Distearoyl-sn-glycero-3-phosphate (Sodium Salt) (DSPA-NA), and combinations thereof.

Non-limiting examples of phosphatidylethanolamines include 1,2-Dierucoyl-sn-glycero-3-phosphoethanolamine (DEPE), 1,2-Dilauroyl-sn-glycero-3-phosphoethanolamine (DLPE), 1,2-Dimyristoyl-sn-glycero-3-phosphoethanolamine (DMPE), 1,2-Dioleoyl-sn-glycero-3-phosphoethanolamine (DOPE), 1,2-Dipalmitoyl-sn-glycero-3-phosphoethanolamine (DPPE), 1,2-Distearoyl-sn-glycero-3-phosphoethanolamine (DSPE), 1-Palm itoyl-2-oleoyl-sn-glycero-3-phosphoethanolamine (POPE), and combinations thereof.

Non-limiting examples of phophatidylglycerols include 1,2-Dierucoyl-sn-glycero-3[Phospho-rac-(1-glycerol . . . ) (Sodium Salt) (DEPG-NA), 1,2-Dilauroyl-sn-glycero-3[Phospho-rac-(1-glycerol . . . ) (Sodium Salt) (DLPG-NA), 1,2-Dilauroyl-sn-glycero-3[Phospho-rac-(1-glycerol . . . ) (Ammonium Salt) (DLPG-NH4, 1,2-Dimyristoyl-sn-glycero-3[Phospho-rac-(1-glycerol . . . ) (Sodium Salt) (DMPG-NA), 1,2-Dimyristoyl-sn-glycero-3[Phospho-rac-(1-glycerol . . . ) (Ammonium Salt) (DMPG-N H4), 1,2-Dimyristoyl-sn-glycero-3[Phospho-rac-(1-glycerol . . . ) (Sodium/Ammonium Salt) (DMPG-NH4/NA), 1,2-Dioleoyl-sn-glycero-3[Phospho-rac-(1-glycerol . . . ) (Sodium Salt) (DOPG-NA), 1,2-Dipalmitoyl-sn-glycero-3[Phospho-rac-(1-glycerol . . . ) (Sodium Salt) (DPPG-NA), 1,2-Dipalmitoyl-sn-glycero-3[Phospho-rac-(1-glycerol . . . ) (Ammonium Salt) (DPPG-NH4), 1,2-Distearoyl-sn-glycero-3[Phospho-rac-(1-glycerol . . . ) (Sodium Salt) (DSPG-NA), 1,2-Distearoyl-sn-glycero-3[Phospho-rac-(1-glycerol . . . ) (Ammonium Salt) (DSPG-NH4), 1-Palm itoyl-2-oleoyl-sn-glycero-3[Phospho-rac-(1-glycerol) . . . ] (Sodium Salt) (POPG-NA), and combinations thereof.

Non-limiting examples of phosphatidylserines include 1,2-Dilauroyl-sn-glycero-3-phosphoserine (Sodium Salt) (DLPS-NA), 1,2-Dimyristoyl-sn-glycero-3-phosphoserine (Sodium Salt) (DMPS-NA), 1,2-Dioleoyl-sn-glycero-3-phosphoserine (Sodium Salt) (DOPS-NA), 1,2-Dipalmitoyl-sn-glycero-3-phosphoserine (Sodium Salt) (DPPS-NA), 1,2-Distearoyl-sn-glycero-3-phosphoserine (Sodium Salt) (DSPS-NA), and combinations thereof.

Non-limiting examples of phosphoinositides include phosphatidylinositol (PI), phosphatidylinositol 4-phosphate (PIP4), phosphatidylinositol 5-phosphate (PIP5), phosphatidylinositol 3,4-bisphosphate (PI(3,4)P2), phosphatidylinositol 3,5-bisphosphate (PI(3,5)P2), phosphatidylinositol 4,5-bisphosphate (PI(4,5)P2), phosphatidylinositol 3,4,5-triphosphate (PI(3,4,5)P3), and combinations thereof.

Non-limiting examples of phosphosphigolipids include ceramide phosphorylcholine (sphingomyelin) (SPH), ceramide phosphorylethanolamine (sphingomyelin) (Cer-PE), ceramide phosphoryllipid, cerebrosides, gangliosides, and combinations thereof.

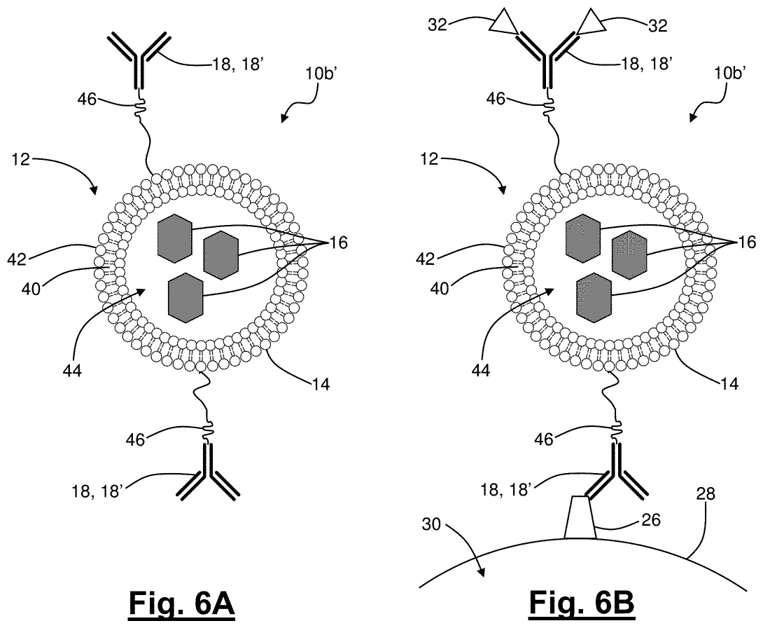

FIG. 6A shows a nanoparticle construct 10b′, which is similar to the nanoparticle construct 10b of FIG. 5, but in which the coupling agent 18 is an antibody or antibody fragment 18′ that binds to a particular cell surface protein or peptide. The antibody or antibody fragment 18′ is optionally linked to the portion of the plurality of lipids 42 by way of the linker 46, which may be a conjugation peptide or PEG (which can have various chain lengths and molecular weights), as non-limiting examples. As described above, in some aspects, the antibody or antibody fragment 18′ is a polyclonal or monoclonal antibody that selectively binds to a protein or peptide that is selectively or not selectively expressed on a cell of interest, such as on a particular cancer cell. Because the cell of interest is isolated from a subject, the nanoparticle construct 10b′ is only coupled to the cell of interest.

FIG. 6B shows the nanoparticle construct 10b′ wherein the antibody or antibody fragment 18′ is bound to a protein or peptide 26 on a surface 28 of an isolated cell 30, which may be a T cell or a CAR-T cell. Here, the isolated cell 30 is magnetically labeled. Optionally, unbound antibodies or antibody fragments 18′ on the nanoparticle construct 10b′ are quenched by a quenching agent 32 to ensure that the unbound antibodies or antibody fragments 18′ do not bind nonspecifically to undesired cells after administering to a subject. As described above, the quenching agent 32 can be a peptide that binds to the antibody or antibody fragment 18′ or a second antibody that that specifically recognizes and binds to the antibody or antibody fragment 18′.

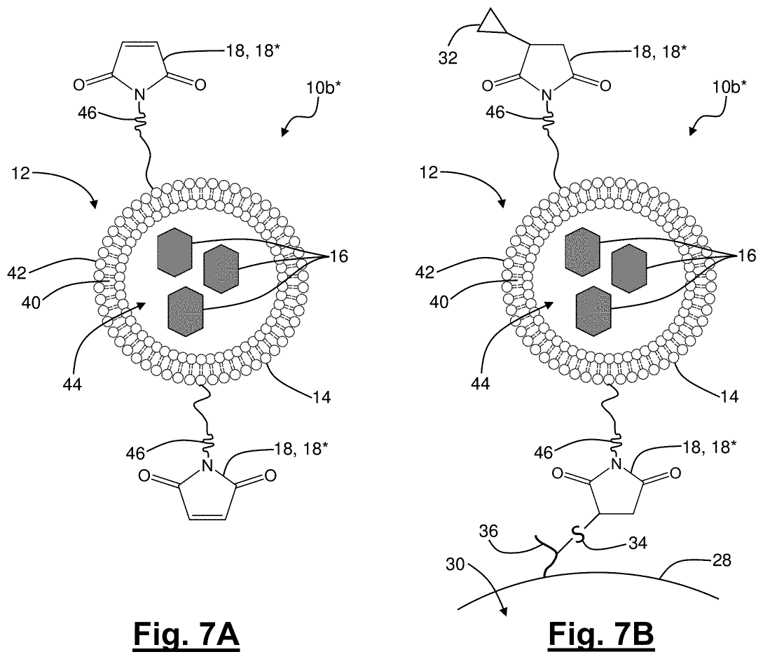

FIG. 7A shows a nanoparticle construct 10b*, which is similar to the nanoparticle construct 10b of FIG. 5, but in which the coupling agent 18 is a maleimide functionality 18* that is optionally linked to the portion of the plurality of lipids 42 by way of the linker 46. For example, the linker 46 may be required to enhance the suitability of the nanoparticle construct 10b*. As a non-limiting example, the linker 46 can be PEG, which can include a variety of chain lengths and molecular weights. Accordingly, the nanoparticle construct 10b* can include lipid-optional linker-maleimide constructs. As discussed above, the maleimide functionality 18* binds to thiol groups present on proteins and peptides that are expressed on cell surfaces. Typically, the thiol groups are present in cysteines present within cell surface proteins or peptides.

FIG. 7B shows the nanoparticle construct 10b* wherein the maleimide functionality 18* is bound to a cysteine thiol 34 found in a protein or peptide 36 extending from the surface 28 of the isolated cell 30, which may be a T cell or a CAR-T cell. Here, the isolated cell 30 is magnetically labeled. Optionally, unbound maleimide functionalities 18* on the nanoparticle construct 10b* are quenched by the quenching agent 32 to ensure that the unbound maleimide functionalities 18* do not bind nonspecifically to undesired cells after administering to a subject. As discussed above, the quenching agent 32 can be a thiolated molecule, such as thiolated PEG, a thiolated amino acid, e.g., thiolated glycine, or combinations thereof, as non-limiting examples.

The nanoparticle constructs 10b′, 10b* of FIGS. 6A and 7A, respectively, are made by a method in accordance with the current technology. The method comprises adding water to a dispersion comprising chloroform, lipids, a coupling agent-functionalized lipid, and the magnetic nanocrystal to form an emulsion. The lipids and the coupling agent-functionalized lipid are combined in a lipid to coupling agent-functionalized lipid ratio of from about 1:2 to about 2:1. The total lipid concentration in the chloroform is greater than or equal to about 3.5 mg/mL to less than or equal to about 4 mg/mL. 200 μL of water is added per mL of the dispersion.

The method then comprises sonicating the emulsion on ice for about 1 minute and adding an additional 6 mL of water to the emulsion per mL of the dispersion. Then, the emulsion is sonicated on ice for about 5 minutes to form a water-in-oil-in-water double emulsion. The method then comprises removing the chloroform, e.g., by evaporation while stirring at about 20° C. under atmospheric pressure for about 6 hours, to form the nanoparticle construct 10b′, 10b*. The nanoparticle construct 10b′, 10b* can be isolated from free lipids by centrifuging through a 60 wt. % sucrose cushion.

An alternative method of making the nanoparticle constructs 10b′, 10b* comprises hydrating a lipid/coupling agent-functionalized lipid film with a magnetic nanocrystal dispersion in phosphate buffered saline (PBS) for about 1 hour with vortexing about every 10 minutes. The hydrated and vortexed dispersion is then subjected to about six freeze (liquid N2)-thaw cycles to form the nanoparticle constructs 10b′ and 10b*, which can be extruded through a polycarbonate filter and purified using a desalting column.

Nanoparticle constructs 10b′, 10b* comprising multi-lamellar liposomes can be made by adding the magnetic nanocrystal to a lipid emulsion comprising a coupling agent-functionalized lipid, while preparing multi-lamellar liposomes using methods known in the art.

Any of the above-described nanoparticle constructs can be coupled to an isolated cell, such as a T cell, by contacting the nanoparticle constructs to the isolated cells. After the nanoparticle constructs are coupled to the cells, unbound nanoparticle constructs are removed from the cells, e.g., by centrifuging and washing. Optionally, free coupling agents remaining on the bound nanoparticle constructs are quenched by contacting the nanoparticle construct-labeled cells with a quenching agent that is appropriate for the coupling agent. The quenched nanoparticle construct-labeled cells can then be separated from the unbound quenching agent, e.g., by centrifuging and washing. In some aspects, the isolated cells are CAR-T cells.

The current technology also provides a method of detecting a cell in a subject. The method comprises subjecting the subject to MRI or MPI, visualizing the cell in a resulting MRI image or MPI image, and determining a location of the cell in the subject from the MRI or MPI image. Prior to the method, the cell is isolated from the subject, coupled to any nanoparticle construct described above, and administered back to the subject. In some aspects, the subject has cancer and the cell is a CAR-T cell or other modified lymphocyte.

The current technology also provides a method of treating a subject in need thereof. The method comprises subjecting the subject, or having the subject subjected to, a treatment comprising isolating a cell from the subject, modifying the cell to generate a therapeutic cell, coupling the therapeutic cell to any nanoparticle construct described above to form a magnetic therapeutic cell, and administering the magnetic therapeutic cell to the subject, for example, intravenously. The method then comprises performing MRI or MPI on the subject, determining the location of the magnetic therapeutic cell within the subject based on a MRI image or a MPI image, and when the location of the magnetic therapeutic cell is determined to be at a location needing the therapeutic cell, continuing the treatment, or when the location of the magnetic therapeutic cell is determined to be at a location other than a location needing the therapeutic cell, discontinuing the treatment. The performing the MRI or MPI can be performed on the subject any time after about 1 day to about 30 days after the treatment. By “having the subject subjected to,” it is meant that the person performing the MRI or MPI and subsequent steps may not have subjected the subject to the treatment, but may have ordered the treatment. In some aspects, the subject has cancer and the cell is a CAR-T cell or other modified lymphocyte.

The current technology further provides another method of treating a subject in need thereof. The method comprises performing MRI or MPI on the subject, wherein the subject is undergoing a treatment in which a cell was isolated from the subject, the cell was modified to generate a therapeutic cell, the therapeutic cell was coupled to the nanoparticle construct to form a magnetic therapeutic cell, and the magnetic therapeutic cell was administered to the subject. The method also comprises determining the location of the magnetic therapeutic cell based on a MRI image or a MPI image and when the location of the magnetic therapeutic cell is determined to be at a location needing the therapeutic cell, continuing the treatment, or when the location of the magnetic therapeutic cell is determined to be at a location other than a location needing the therapeutic cell, discontinuing the treatment. In some aspects, the subject has cancer and the cell is a CAR-T cell or other modified lymphocyte.

The current technology yet further provides another method of treating a subject in need thereof. The method comprises administering or having administered a first test dose of magnetic therapeutic cells prepared by any method or combination of methods described herein. Within a week or two weeks of the administering, the method comprises performing MRI or MPI on the subject, determining the location of the magnetic therapeutic cell based on a MRI image or a MPI image, and when the location of the magnetic therapeutic cell is determined to be at a location needing the therapeutic cell, administering a second dose of the magnetic therapeutic cells to the subject, where the second dose is larger than the first test dose. When the location of the magnetic therapeutic cell is determined to be at a location other than a location needing the therapeutic cell, the method comprises subjecting the subject to a different treatment. In some aspects, the subject has cancer and the cell is a CAR-T cell or other modified lymphocyte.

Embodiments of the present technology are further illustrated through the following non-limiting example.

### EXAMPLE

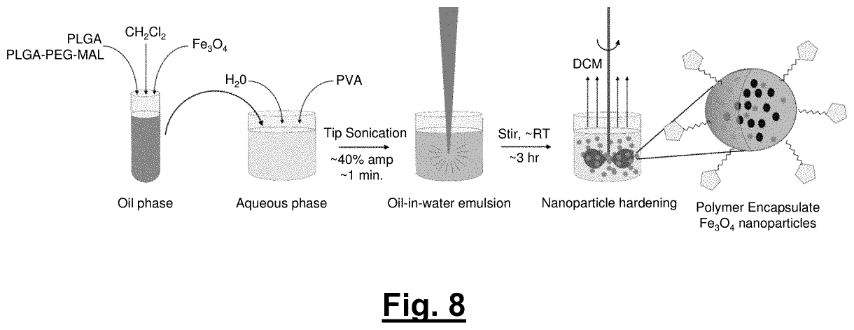

Nanoparticle constructs are synthesized by an oil-in-water emulsion method, as shown in FIG. 8. For batches of nanoparticle constructs, 8 nm magnetite nanocrystals in 0.3 ml dichloromethane are added to an oil phase, which is PLGA only; a 200 mg mixture of a 10:1 mixture of PLGA and PLGA-PEG-maleimide dissolved in 5 ml dichloromethane; and a mixture of PLGA, PLGA-fluorescein isothiocyanate (FITC), and PLGA-PEG-maleimide. PEG is an FDA approved polymer useful for preventing non-specific uptake of particles by un-targeted cells, and maleimide is a reactive group at the end of the polymer that can react with thiol groups (—SH) in cysteine in the surface proteins of cells to form covalent bonds. Batches of nanoparticle constructs are synthesized with 10% iron oxide, 25% iron oxide, 50% iron oxide, and 100% iron oxide relative to the total polymer.

The oil phase is then poured into a solution of PVA (50 ml, 2% PVA w/v) and tip sonicated or homogenized. The resultant emulsion stirs for 3 hours at 37° C. to ensure complete evaporation of the dichloromethane. The hardened particles are the nanoparticle constructs, which are collected by centrifugation, washed four times with deionized water, and frozen and lyophilized. As shown in Table 1, mean particle size varies from 100-1000 nm, with mean interval sizes of 100 nm and particle size varying based on the energy applied during tip sonication or use of a high speed homogenizer. Surface maleimide group density is about 10%. Iron content is about 50% w/w.

TABLE 1
Characteristics of nanoparticle constructs made by five different recipes.

| Desired Size | Obtained size | Polydispersity | Zeta potential | |
|---|---|---|---|---|
| (nm) | (nm) | <1 | (mV) | Recipe |
| 200 | 213 | 0.235 | −17.7 | Tip-sonicated: 60 s, 40%, 20 kHz |
| 450 | 477.7 | 0.462 | −14.8 | Homogenized: 30k rpm, 480 s |
| 600 | 608.9 | 0.411 | −19.7 | Homogenized: 30k rpm, 70 s |
| 750 | 720.1 | 0.506 | −19.4 | Homogenized: 30k rpm, 40 s |
| 1000 | 1226.5 | 0.895 | −11.7 | Homogenized: 30k rpm, 30 s |

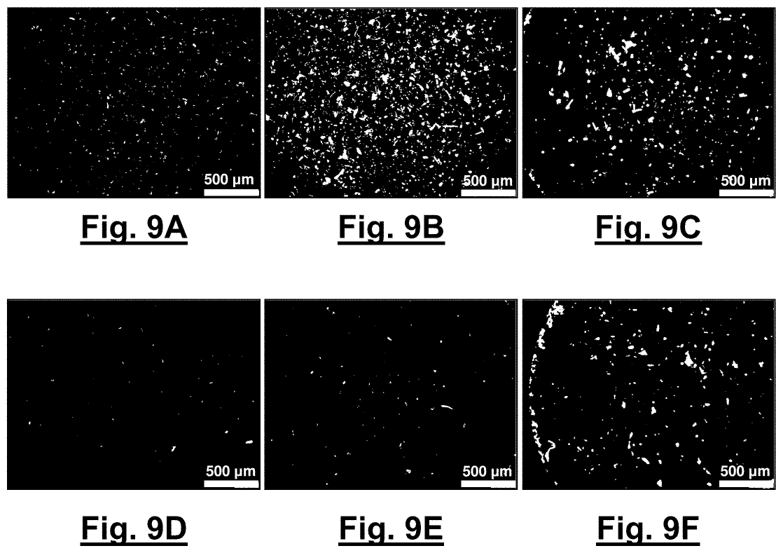

As discussed above, FITC-labeled thiolated PEG (FITC-PEG-SH) is synthesized. The nanoparticle constructs are contacted with the FITC-PEG-SH, whereby the maleimide functionalities of the nanoparticle constructs form covalent bonds with the thiol (SH) as the FITC-PEG-S-nanoparticle constructs. After washing away unbound FITC-PEG-SH, the FITC-PEG-S-nanoparticle constructs are visualized by fluorescence microscopy. FIGS. 9A, 9B, and 9C show resulting fluorescence micrographs, in which the FITC-PEG-S-nanoparticle constructs each comprise 10% iron oxide and 10% maleimide, 50% maleimide, and 100% maleimide, respectively. As can be seen in the micrographs, fluorescence intensity appears to diminish with iron oxide concentration (likely due to light blockage). FIGS. 9D, 9E, and 9F show resulting fluorescence micrographs, in which the FIGC-PEG-S-nanoparticle constructs each comprise 25% iron oxide and 10% maleimide, 50% maleimide, and 100% maleimide, respectively. Fluorescence quenching is directly proportional to iron oxide content in the nanoparticle constructs. The fluorescence micrographs show that the nanoparticle constructs are successfully bound to the FITC-PEG-SH.

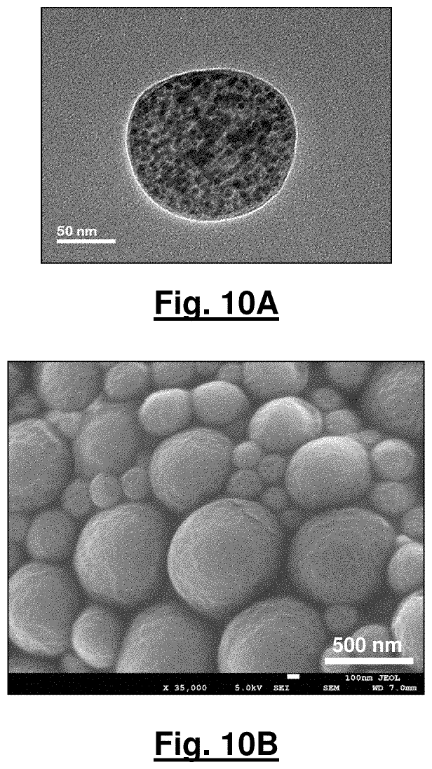

FIG. 10A shows a transmission electron microscopy (TEM) micrograph of a representative nanoparticle construct comprising 50% iron oxide and 10% maleimide (the scale bar is 50 nm). FIG. 10B shows a scanning electron microscopy (SEM) micrograph of a representative nanoparticle construct comprising 50% iron oxide, 10% maleimide, and 20% FITC (the scale bar is 500 nm).

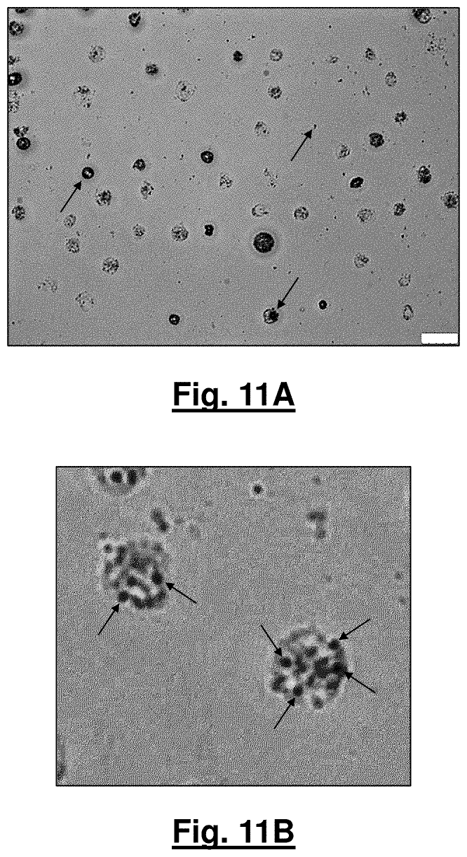

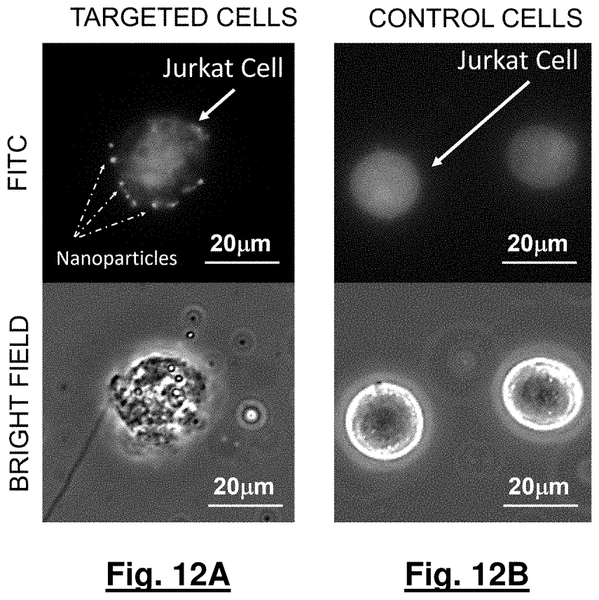

The nanoparticle constructs suspended in Milli-Q® water are contacted with Jurkat cells (T cells) suspended in serum-free RPMI. More particularly, Jurkat cells are incubated with nanoparticle constructs at ratios of 50, 200, and 500 nanoparticle constructs per cell in a 96-well plate. After 30 minutes, the cells are washed and the nanoparticle construct binding is analyzed using bright field and fluoresce microscopy. FIGS. 11A and 11B show bright field micrographs of the cells under increasing magnification. The black dots, some of which are pointed to by arrows, are the nanoparticle constructs bound to the Jurkat cells. These bright field micrographs show that the nanoparticle constructs are successfully bound to the Jurkat cells. FIG. 12A shows epifluorescent and bright field microscopy images of targeted Jurkat cells with conjugated nanoparticle constructs on the surface. FIG. 12B shows epifluorescent and bright field microscopy images of control Jurkat cells incubated with similar nanoparticle constructs, but without maleimide.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A nanoparticle construct comprising:
a nanoparticle defining an outer surface;
a magnetic nanocrystal carried by the nanoparticle; and
a coupling agent extending from the outer surface of the nanoparticle,
wherein the coupling agent is configured to couple the nanoparticle construct to a cell.

2. The nanoparticle construct according to claim 1, wherein the nanoparticle comprises a polymer matrix, and wherein the magnetic nanocrystal is embedded within the polymer matrix.

3. The nanoparticle construct according to claim 2, wherein the coupling agent is a maleimide functionality, an antibody, an antibody fragment, or combinations thereof.

4. The nanoparticle construct according to claim 3, wherein the polymer matrix comprises poly(lactic co-glycolic acid) (PLGA) and the coupling agent comprises the maleimide functionality, wherein the maleimide functionality is coupled to the PLGA by way of a linker.

5. The nanoparticle construct according to claim 4, wherein the linker comprises polyethylene glycol (PEG).

6. The nanoparticle construct according to claim 5, wherein the nanoparticle construct is coupled to a T cell by way of a bond between the maleimide functionality and a thiol group on the T cell's cell membrane.

7. A method of synthesizing the nanoparticle construct according to claim 2, the method comprising:
dissolving a polymer and the coupling agent in an organic solvent to form a polymer solution;
adding a dispersion comprising the magnetic nanocrystal to the polymer solution to form a precursor solution;
transferring the precursor solution to a solution comprising poly-vinyl alcohol (PVA) and water to form a nanoparticle precursor solution;
sonicating or homogenizing the nanoparticle precursor solution; and
removing the organic solvent to form the nanoparticle construct.

8. The nanoparticle construct according to claim 1, wherein the nanoparticle comprises a substantially spherical lipid membrane comprising a plurality of lipids that define an interior compartment, the magnetic nanocrystal is disposed within the interior compartment, and the coupling agent is bonded to at least one lipid of the plurality.

9. The nanoparticle construct according to claim 8, wherein the coupling agent is a maleimide functionality, an antibody, an antibody fragment, or combinations thereof.

10. The nanoparticle construct according to claim 9, wherein the nanoparticle construct is coupled to a T cell by way of the coupling agent.

11. The nanoparticle construct according to claim 10, wherein the coupling agent comprises the maleimide functionality, and wherein the maleimide functionality is bonded to a thiol group on the T cell's cell membrane.

12. A method of synthesizing the nanoparticle construct according to claim 8, the method comprising:
adding water to a dispersion comprising chloroform, lipids, at least one coupling agent-functionalized lipid, and the magnetic nanocrystal to form an emulsion;
sonicating the emulsion;
adding additional water to the emulsion and sonicating to form a water-in-oil-in-water double emulsion; and
removing the chloroform from the water-in-oil-in-water double emulsion to form the nanoparticle construct.

13. A method of detecting a cell in a subject, the method comprising:
subjecting the subject to magnetic resonance imaging (MRI) or magnetic particle imaging (MPI);
visualizing the cell in a resulting MRI scan or MPI scan; and
determining a location of the cell in the subject,
wherein the cell was previously isolated from the subject, coupled to the nanoparticle construct according to claim 1, and administered back to the subject.

14. The method according to claim 13, wherein the cell is a T cell.

15. The method according to claim 14, wherein the subject has cancer and the T cell is a T cell comprising a chimeric antigen receptor (CAR-T cell).

16. A method of treating a subject in need thereof, the method comprising:
subjecting the subject, or having the subject subjected to, a treatment comprising:
isolating a cell from the subject;
modifying the cell to generate a therapeutic cell;
coupling the therapeutic cell to a nanoparticle construct to form a magnetic therapeutic cell, the nanoparticle construct comprising:
a nanoparticle defining an outer surface;
a magnetic nanocrystal carried by the nanoparticle; and
a coupling agent extending from the outer surface of the nanoparticle,
wherein the coupling agent is configured to couple the nanoparticle construct to the therapeutic cell; and
administering the magnetic therapeutic cell to the subject;
performing magnetic resonance imaging (MRI) or magnetic particle imaging (MPI) on the subject;
determining the location of the magnetic therapeutic cell based on a MRI image or a MPI image; and
when the location of the magnetic therapeutic cell is determined to be at a location needing the therapeutic cell, continuing the treatment; or
when the location of the magnetic therapeutic cell is determined to be at a location other than a location needing the therapeutic cell, discontinuing the treatment.

17. The method according to claim 16, wherein the nanoparticle comprises either a polymer matrix or a substantially spherical lipid membrane defining an interior compartment carrying the magnetic nanocrystal.

18. The method according to claim 16, wherein the coupling agent is a maleimide functionality, an antibody, an antibody fragment, or combinations thereof.

19. A method of treating a subject in need thereof, the method comprising:
performing magnetic resonance imaging (MRI) or magnetic particle imaging (MPI) on the subject, wherein the subject is undergoing a treatment in which:
a cell was isolated from the subject;
the cell was modified to generate a therapeutic cell;
the therapeutic cell was coupled to a nanoparticle construct to form a magnetic therapeutic cell, the nanoparticle construct comprising:
a nanoparticle defining an outer surface;
a magnetic nanocrystal carried by the nanoparticle; and
a coupling agent extending from the outer surface of the nanoparticle and bonded to the therapeutic cell; and
the magnetic therapeutic cell was administered to the subject;
determining the location of the magnetic therapeutic cell based on a MRI image or a MPI image; and
when the location of the magnetic therapeutic cell is determined to be at a location needing the therapeutic cell, continuing the treatment; or
when the location of the magnetic therapeutic cell is determined to be at a location other than a location needing the therapeutic cell, discontinuing the treatment.

20. The method according to claim 19, wherein the nanoparticle comprises either a polymer matrix or a substantially spherical lipid membrane defining an interior compartment carrying the magnetic nanocrystal, and wherein the coupling agent is a maleimide functionality, an antibody, an antibody fragment, or combinations thereof.
